(19) United States
(12) Patent Application Publication
(10) Pub. No.: US 2024/0284950 A1
(43) Pub. Date: Aug. 29, 2024

(54) SYSTEMS AND METHODS FOR PREPARING COMPOUND CRYSTALS OF ERYTHRITOL AND HIGH-INTENSITY SWEETENERS

(71) Applicant: ZHEJIANG HUAKANG PHARMACEUTICAL CO., LTD., Quzhou (CN)

(72) Inventors: Shufang QIN, Quzhou (CN); Mian LI, Quzhou (CN); Wenyao ZHANG, Quzhou (CN); Wulong YANG, Quzhou (CN); Qiang WU, Quzhou (CN); Ni ZHEN, Quzhou (CN); Weidong XU, Quzhou (CN)

(73) Assignee: ZHEJIANG HUAKANG PHARMACEUTICAL CO., LTD., Quzhou (CN)

(21) Appl. No.: 18/651,637

(22) Filed: Apr. 30, 2024

Related U.S. Application Data
(63) Continuation of application No. PCT/CN2023/096372, filed on May 25, 2023.

(30) Foreign Application Priority Data
Dec. 25, 2022 (CN) 202211669958.0

(51) Int. Cl. A23L 27/30

(52) U.S. Cl. CPC A23L 27/34

(57) ABSTRACT

The present disclosure discloses systems and methods for preparing compound crystals of erythritol and high-intensity sweeteners. The systems may comprise a fermentation liquid storage tank, a ceramic membrane filtration system, a nanofiltration system, a first evaporator, a first crystallization kettle, a first centrifuge, a sugar dissolution tank, a decolorization tank, an ion exchange column, a blending tank, a second evaporator, a second crystallization kettle, a second centrifuge, a hot-air drying tank, a fluidized drying bed, and a finished product tank sequentially connected through pipelines. The systems may further comprise a high-intensity sweetener storage tank, a primary mother liquor tank, and a secondary mother liquor tank. The finished product tank may store a prepared compound crystallization product of the erythritol and the high-intensity sweetener. The particle size of the compound crystallization product obtained according to the present disclosure is concentrated and controllable, thereby reducing the preparation cost.

### CROSS-REFERENCE TO RELATED APPLICATIONS

This application is a Continuation of International Application No. PCT/CN2023/096372, filed on May 25, 2023, which claims priority to Chinese Patent Application No. 202211669958.0, filed on Dec. 25, 2022, the entire contents of each of which are hereby incorporated by reference.

### TECHNICAL FIELD

The present disclosure generally relates to the technical field of sugar alcohol preparation, and more particularly, to systems and methods for preparing compound crystals of erythritol and high-intensity sweeteners.

### BACKGROUND

Erythritol is a natural sugar alcohol sweetener with many advantages, such as pure sweetness, a low calorific value, non-cariogenic property, refreshing entrance, etc. Since the sweetness of the erythritol is 60-70% of the sweetness of sucrose, compared with the sucrose, the sweetness of the erythritol is unspectacular, the taste of the erythritol is thin, and the erythritol has a delayed bitterness. Therefore, in order to cover up these defects when the erythritol is used alone, and satisfy people's requirements for good taste and low calorie, high-intensity sweeteners or other sugar alcohols are often used to be compounded or co-crystallized with the erythritol in the industry.

At present, processing manners, which include performing coating spray on the surface of the erythritol by using a spray granulation technique, directly using a mixed sugar solution of the erythritol and other high-intensity sweeteners for spray granulation, etc., have been adopted. These processing manners have problems, such as coating falling off, uneven taste, etc. Alternatively, a co-crystallization product is obtained by using a co-crystallization technique to perform a cooling crystallization or melt crystallization on the erythritol and other high-intensity sweeteners. For example, in the Chinese Patent Application No. CN110179096A, a compound sweetener was obtained by performing a liquid cooling crystallization on the erythritol, inulin, stevioside, and mogroside for compound crystallization. The particle size of the compound sweetener is consistent, and the taste of the compound sweetener is good. In the Chinese Patent Application No. CN103262972A, co-crystallization crystals were obtained by performing the melt crystallization on the erythritol and sucralose for co-crystallization. The co-crystallization crystals have a good instant solubility and a uniform sweetness. However, none of the manners, whether using the cooling crystallization or the melt crystallization, disclose whether the particle sizes of products of the compound crystals can be regulated, which is not conducive to the preparation of the products with different particle size specifications. If the particle size specification of the products is required, the products prepared by the existing manners can only be sieved through a sieving treatment to obtain the products with the required particle size, which increases a preparation cost.

### SUMMARY

The technical problem to be solved by the present disclosure is to provide a system and a method for preparing a compound crystal of erythritol and a high-intensity sweetener. The compound crystal of the erythritol and the high-intensity sweetener can be obtained by using an evaporation crystallization. By using a regulatory manner that coordinates an added particle size of erythritol crystal seeds and a stirring speed, the particle size of the obtained compound crystals is concentrated and controllable, which is conducive to the preparation of products with different particle size specifications, thereby reducing the preparation cost. At the same time, the flavor of the erythritol can be improved, and the taste of the erythritol can be uniform, which is more consistent with consumer preferences.

The present disclosure provides the system for preparing the compound crystal of the erythritol and the high-intensity sweetener. The system may comprise a fermentation liquid storage tank, a ceramic membrane filtration system, a nanofiltration system, a first evaporator, a first crystallization kettle, a first centrifuge, a sugar dissolution tank, a decolorization tank, an ion exchange column, a blending tank, a second evaporator, a second crystallization kettle, a second centrifuge, a hot-air drying tank, a fluidized drying bed, and a finished product tank sequentially connected through pipelines. The system may further comprise a high-intensity sweetener storage tank, a primary mother liquor tank, and a secondary mother liquor tank. The finished product tank may store a prepared compound crystallization product of the erythritol and the high-intensity sweetener.

The present disclosure provides the method for preparing the compound crystal of the erythritol and the high-intensity sweetener. The method may use the system for preparing the compound crystal of the erythritol and the high-intensity sweetener. The method may include the following operations.

Operation 1, a penetrating liquid may be obtained by sequentially conveying the sterilized erythritol fermentation liquid stored in the fermentation liquid storage tank to the ceramic membrane filtration system for filtration and the nanofiltration system for separation, and a solid erythritol monocrystalline sugar and a liquid primary mother liquor may be obtained, respectively, by conveying the penetrating liquid to the first evaporator for concentration, the first crystallization kettle for crystallization, and the first centrifuge for centrifugation through the pipelines. The primary mother liquor may be temporarily stored in the primary mother liquor tank and then returned to the first evaporator through the pipeline for reuse. In operation 1, a content of the erythritol in the penetrating liquid after the separation performed by the nanofiltration system may be larger than 93%, and a light transmittance of the erythritol in the penetrating liquid may be larger than 85%.

Operation 2, a dissolved liquid may be obtained by dissolving the erythritol monocrystalline sugar in the dissolving sugar tank, a decolorization operation through the decolorization tank and an impurity removal operation through the ion exchange column may be performed on the dissolved liquid sequentially, a blending liquid may be obtained by mixing the processed dissolved liquid with the high-intensity sweetener liquid conveyed by the high-intensity sweetener storage tank and a secondary mother liquor conveyed by the secondary mother liquor tank, and then a solid compound moist sugar of the erythritol and the high-intensity sweetener, and the liquid secondary mother liquor may be obtained, respectively, by sequentially introducing the blending liquid into the second evaporator for concentration, the second crystallization kettle for crystallization, and the second centrifuge for centrifugal separation through the pipelines. The secondary mother liquor may be temporarily stored in the secondary mother liquor tank and then returned to the blending tank through the pipeline for reuse. In operation 2, a light transmittance of the processed dissolved liquid may be larger than 97% and a conductivity of the processed dissolved liquid may be less than 30 microsecond per centimeter (μs/cm). A refractive index of a blending concentrate after the concentration is performed on the blending liquid by the second evaporator may be within a range from 65 to 70%, an erythritol content of the blending concentrate may be within a range from 95 to 99.9%, and a high-intensity sweetener content of the blending concentrate may be within a range from 0.1 to 5%.

Operation 3, the prepared compound crystallization product of the erythritol and the high-intensity sweetener temporarily stored in the finished product tank may be obtained by sequentially performing a hot-air drying operation through the hot-air drying tank and a cold-air drying operation through the fluidized drying bed on the obtained compound moist sugar of the erythritol and the high-intensity sweetener through the pipelines.

The system and the method for preparing the compound crystal of the erythritol and the high-intensity sweetener of the present disclosure have the following features.

1. The particle size of the compound crystal of the erythritol and the high-intensity sweetener obtained according to the present disclosure is concentrated and controllable.

2. A shape of the compound crystal of the erythritol and the high-intensity sweetener obtained according to the present disclosure is similar to that of an erythritol crystal, and a crystal form of the compound crystal is relatively good.

3. The sweetness of the compound crystal of the erythritol and the high-intensity sweetener obtained according to the present disclosure may be adjustable, which is within a range of 0.7 to 2 times of the sweetness of sucrose, and the taste of the compound crystal is uniform and better than that of the erythritol crystal.

One or more embodiments of the present disclosure provide a non-transitory computer-readable storage medium storing computer instructions. When a computer reads the computer instructions from the storage medium, the computer may execute the method for preparing the compound crystal of the erythritol and the high-intensity sweetener.

### BRIEF DESCRIPTION OF THE DRAWINGS

This present disclosure will be further illustrated by way of exemplary embodiments, which will be described in detail through the accompanying drawings. These embodiments are not limiting, in these embodiments the same numbering indicates the same structure, wherein:

FIG. 1 is a schematic diagram illustrating a system for preparing a compound crystal erythritol and a high-intensity sweetener according to some embodiments of the present disclosure;

FIG. 2 is a flowchart illustrating an exemplary process for generating and sending preparation instructions according to some embodiments of the present disclosure;

FIG. 3 is a schematic diagram illustrating an exemplary process for determining setting values of preparation parameters through a parameter prediction model according to some embodiments of the present disclosure; and

FIG. 4 is a schematic diagram illustrating a particle size distribution for preparing a compound crystallization product of erythritol and a high-intensity sweetener according to some embodiments of the present disclosure.

### DETAILED DESCRIPTION

In order to more clearly illustrate the technical problems, the technical solutions, and beneficial effects of the embodiments of the present disclosure, the accompanying drawings to be used in the description of the embodiments will be briefly described below. It should be understood that the specific embodiments described herein are merely for illustrating the present disclosure and are not intended to limit the present disclosure.

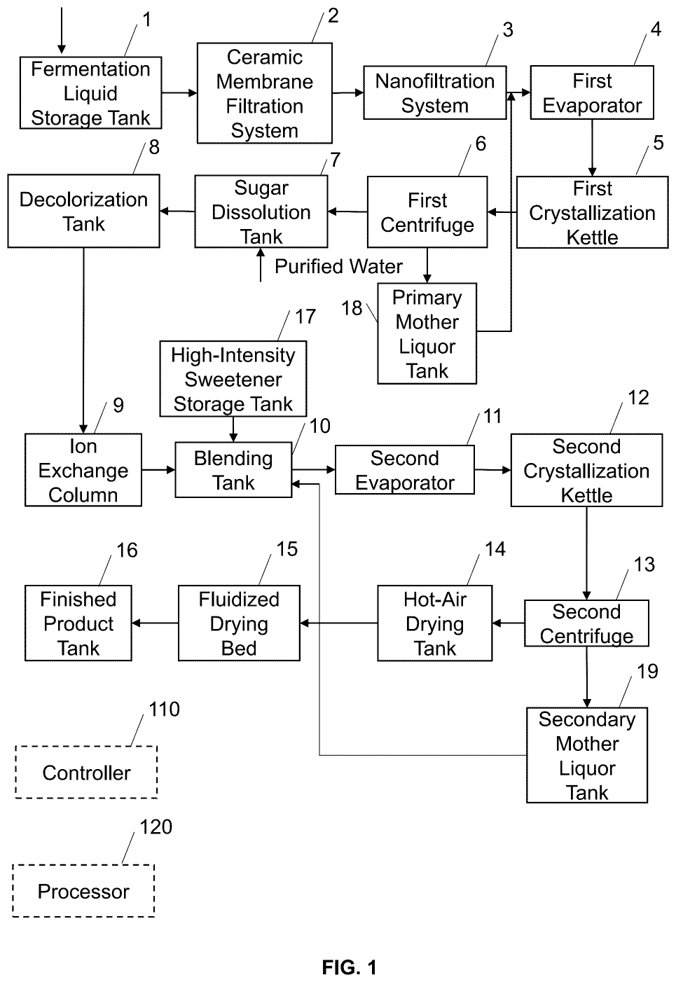

FIG. 1 is a schematic diagram illustrating a system for preparing a compound crystal erythritol and a high-intensity sweetener according to some embodiments of the present disclosure.

Referring to FIG. 1, the system for preparing the compound crystal of the erythritol and the high-intensity sweetener may include a fermentation liquid storage tank 1, a ceramic membrane filtration system 2, a nanofiltration system 3, a first evaporator 4, a first crystallization kettle 5, a first centrifuge 6, a sugar dissolution tank 7, a decolorization tank 8, an ion exchange column 9, a blending tank 10, a second evaporator 11, and a second crystallization kettle 12, a second centrifuge 13, a hot-air drying tank 14, a fluidized drying bed 15, and a finished product tank 16 sequentially connected through pipelines. The system may further include a high-intensity sweetener storage tank 17, a primary mother liquor tank 18, and a secondary mother liquor tank 19. Directions of arrows in the FIG. 1 indicate flow directions of materials in the system.

The fermentation liquid storage tank 1 is disposed with a feed inlet for a sterilized erythritol fermentation liquid, the nanofiltration system 3 is disposed with a penetrating liquid feed inlet, and the penetrating liquid feed inlet is connected to a feed inlet of the first evaporator 4 through a pipeline. The sugar dissolution tank 7 is disposed with a feed inlet for purified water, and the blending tank 10 is disposed with a feed inlet for a high-intensity sweetener.

Each of the first centrifuge 6 and the second centrifuge 13 is disposed with a solid material feed outlet and a liquid material feed outlet. The liquid material feed outlet of the first centrifuge 6 is connected to a feed inlet of the primary mother liquor tank 18 through the pipeline, and a feed outlet of the primary mother liquor tank 18 is connected to a feed inlet of the first evaporator 4 through a pipeline. The liquid material feed outlet of the second centrifuge 13 is connected to a feed inlet of the secondary mother liquor tank 19 through a pipeline, and a feed outlet of the secondary mother liquor tank 19 is connected to a feed inlet of the blending tank 10 through a pipeline. The finished product tank stores a prepared compound crystallization product of the erythritol and the high-intensity sweetener.

The sterilized erythritol fermentation liquid enters the fermentation liquid storage tank 1 through the feed inlet for temporary storage. The sterilized erythritol fermentation liquid is filtered by the ceramic membrane filtration system 2 and separated by the nanofiltration system 3, respectively, to remove most of impurities and obtain a penetrating liquid, and then an erythritol monocrystalline sugar and a primary mother liquor are obtained by sequentially conveying the penetrating liquid to the first evaporator 4 for evaporation and concentration, the first crystallization kettle 5 for crystallization, and the first centrifuge 6 for centrifugation through the pipelines. A dissolved liquid is obtained by conveying the erythritol monocrystalline sugar to the dissolving sugar tank 7 through the pipeline and dissolving the erythritol monocrystalline sugar in the dissolving sugar tank 7. A decolorization operation through the decolorization tank 8 and an impurity removal operation through the ion exchange column 9 are performed on the dissolved liquid sequentially. A blending liquid is obtained by mixing the processed dissolved liquid with the high-intensity sweetener liquid conveyed by the high-intensity sweetener storage tank 17 and a secondary mother liquor conveyed by the secondary mother liquor tank 19 in the blending tank 10, and then the compound crystallization product of the erythritol and the high-intensity sweetener is obtained by sequentially introducing the blending liquid into the second evaporator 11 for concentration, the second crystallization kettle 12 for crystallization, the second centrifuge 13 for centrifugal separation, the hot-air drying tank 14 for the hot-air drying, and the fluidized drying bed 15 for the cold-air drying.

The mother liquor is a remained saturated liquid after crystals have been separated during the crystallization process. The primary mother liquor is a retained mother liquor after a first crystallization, which may also be referred to as a primary centrifugal mother liquor. The secondary mother liquor is a retained mother liquor after a secondary crystallization, wherein the secondary crystallization refers to that a crystallization is performed on a liquid obtained after the primary mother liquor is concentrated and decolorized to remove impurities. The secondary mother liquor may also be referred to as a secondary centrifugal mother liquor.

The high-intensity sweetener includes at least one of stevioside, mogroside, sucralose, acesulfame, or aspartame.

The present disclosure also discloses a method for preparing a compound crystal of erythritol and a high-intensity sweetener. The method uses the system for preparing the compound crystal of the erythritol and the high-intensity sweetener. The method may include the following operations.

In operation 1, a penetrating liquid may be obtained by sequentially conveying the sterilized erythritol fermentation liquid stored in the fermentation liquid storage tank 1 to the ceramic membrane filtration system 2 for filtration and the nanofiltration system 3 for separation, and a solid erythritol monocrystalline sugar and a liquid primary mother liquor may be obtained, respectively, by conveying the penetrating liquid to the first evaporator 4 for concentration, the first crystallization kettle 5 for crystallization, and the first centrifuge 6 for centrifugation through the pipelines. The primary mother liquor may be temporarily stored in the primary mother liquor tank 18 and then returned to the first evaporator 4 through the pipeline for reuse. In operation 1, a content of the erythritol in the penetrating liquid after the separation performed by the nanofiltration system 3 is larger than 93%, and a light transmittance of the erythritol in the penetrating liquid is larger than 85%.

The content of the erythritol refers to a weight percentage of the erythritol contained in the penetrating liquid.

The light transmittance may reflect a capacity of light to pass through the penetrating liquid. In some embodiments, the light transmittance may be expressed as a percentage. The larger the capacity of light to pass through the penetrating liquid is, the larger the value of the light transmittance may be.

In operation 2, a dissolved liquid may be obtained by dissolving the erythritol monocrystalline sugar in the dissolving sugar tank 7. A decolorization operation through the decolorization tank 8 and an impurity removal operation through the ion exchange column 9 may be performed on the dissolved liquid sequentially. A blending liquid may be obtained by mixing the processed dissolved liquid with the high-intensity sweetener liquid conveyed by the high-intensity sweetener storage tank 17 and a secondary mother liquor conveyed by the secondary mother liquor tank 19. Then a solid compound moist sugar of the erythritol and the high-intensity sweetener, and the liquid secondary mother liquor may be obtained, respectively, by sequentially introducing the blending liquid into the second evaporator 11 for concentration, the second crystallization kettle 12 for crystallization, and the second centrifuge 13 for centrifugal separation through the pipelines. The secondary mother liquor may be temporarily stored in the secondary mother liquor tank 19 and then returned to the blending tank 10 through the pipeline for reuse. In operation 2, a light transmittance of the processed dissolved liquid is larger than 97% and a conductivity of the processed dissolved liquid is less than 30 μs/cm. A refractive index of a blending concentrate after the concentration is performed on the blending liquid by the second evaporator is within a range from 65 to 70%, an erythritol content of the blending concentrate is within a range from 95 to 99.9%, and a high-intensity sweetener content of the blending concentrate is within a range from 0.1 to 5%.

The conductivity may reflect a capacity of the processed dissolved liquid to conduct electricity. The larger the capacity of the processed dissolved liquid to conduct electricity is, the higher the conductivity of the processed dissolved liquid is.

The refractive index refers to a ratio of a speed at which light travels through air to a speed at which light travels through the blending concentrate. The refractive index may reflect a composition of a liquid mixture of the blending concentrate and a purity of a liquid substance of the blending concentrate. In some embodiments, the refractive index may be determined from a ratio of a sine value of an incidence angle to a sine value of a refractive index angle of the light entering the blending concentrate.

The high-intensity sweetener content refers to a weight percentage of the high-intensity sweetener contained in the blending concentrate.

In operation 3, the prepared compound crystallization product of the erythritol and the high-intensity sweetener temporarily stored in the finished product tank 16 may be obtained by sequentially performing a hot-air drying operation through the hot-air drying tank 14 and a cold-air drying operation through the fluidized drying bed 15 on the obtained compound moist sugar of the erythritol and the high-intensity sweetener through the pipelines.

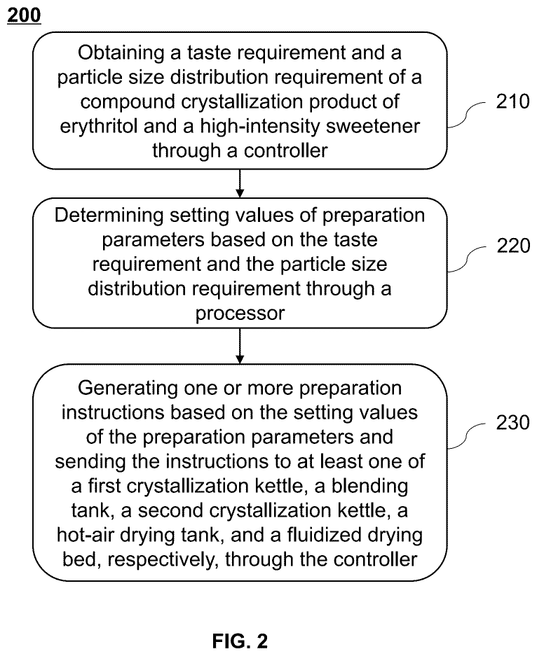

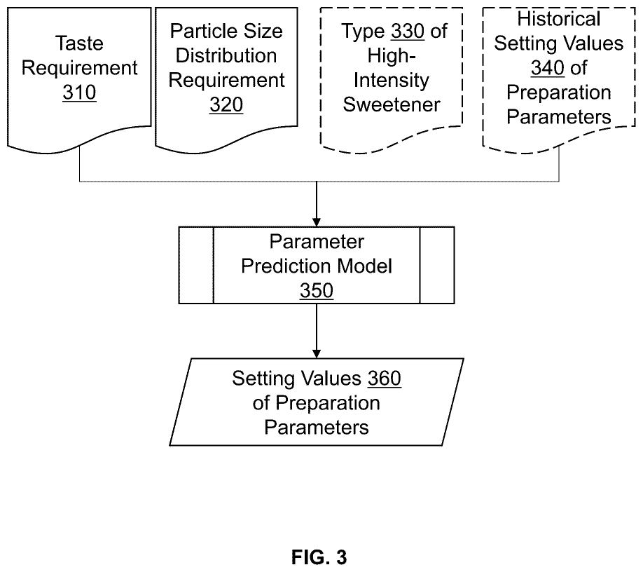

The preparation process may include a plurality of preparation parameters. The preparation parameters refer to relevant parameters required during the preparation of the product. In some embodiments, the preparation parameters may include a first crystallization temperature of the first crystallization kettle 5, a blending ratio of the blending tank 10, a stirring speed of the second crystallization kettle 12, a second crystallization temperature of the second crystallization kettle 12, a pressure of the second crystallization kettle 12, an addition ratio and a crystal seed particle size of erythritol crystal seeds in the second crystallization kettle 12, a crystal-cultivation time length and an evaporation-crystallization time length in the second crystallization kettle 12, a hot-air temperature in the hot-air drying tank 14, a cold-air temperature in the fluidized drying bed 15, or the like, or any combination thereof. In some embodiments, setting values of the preparation parameters may be determined based on actual requirement(s). The setting values of the preparation parameters refer to values corresponding to the preparation parameters set in an actual preparation process. For example, the setting values of the preparation parameters may be determined based on a taste requirement and a particle size distribution requirement. As another example, the setting values of the preparation parameters may be determined through a parameter prediction model. More descriptions regarding the determination of the setting values of the preparation parameters may be found in FIGS. 2 and 3, and relevant descriptions thereof.

The first crystallization temperature of the first crystallization kettle 5 refers to a temperature in the first crystallization kettle 5 when the crystallization occurs in an erythritol concentrate in the first crystallization kettle 5, wherein the erythritol concentrate is obtained after the concentration is performed on the penetrating liquid by the first evaporator 4. The first crystallization temperature may be within a temperature range. In some embodiments, the temperature range of the first crystallization temperature may be set according to actual requirement(s).

The blending ratio of the blending tank refers to a blending ratio of the high-intensity sweetener liquid and the processed dissolved liquid after the decolorization operation and the impurity removal operation, or a blending ratio of the processed dissolved liquid, the high-intensity sweetener liquid, and the secondary mother liquor. The blending ratio of the blending tank may be within a ratio range. In some embodiments, the blending ratio of the blending tank may be set according to actual requirement(s).

In some embodiments, in operation 1, a refractive index of the erythritol concentrate obtained after the concentration is performed on the penetrating liquid by the first evaporator 4 is within a range from 55 to 60%, and a crystallization temperature of the first crystallization kettle 5 is within a range from 60 to 65° C.

The stirring speed of the second crystallization kettle 12 refers to a speed at which the blending concentrate is stirred in the second crystallization kettle 12. The stirring speed of the second crystallization kettle 12 may be within a speed range. In some embodiments, the stirring speed of the second crystallization kettle 12 may be set according to actual requirement(s).

The second crystallization temperature of the second crystallization kettle 12 refers to a temperature in the second crystallization kettle 12 when the crystallization occurs in the blending concentrate in the second crystallization kettle 12. The second crystallization temperature of the second crystallization kettle 12 may be within a temperature range. In some embodiments, the second crystallization temperature may be set according to actual requirement(s).

The pressure of the second crystallization kettle 12 refers to a pressure in the second crystallization kettle 12 when the crystallization occurs in the blending concentrate in the second crystallization kettle 12. The pressure of the second crystallization kettle 12 may be within a pressure range. In some embodiments, the pressure of the second crystallization kettle may be set according to actual requirement(s).

The addition ratio of the erythritol crystal seeds refers to a mass ratio of the erythritol crystal seeds added in the second crystallization kettle 12 to the blending concentrate. The addition ratio of the erythritol crystal seeds may be within a percentage range. In some embodiments, the addition ratio of the erythritol crystal seeds may be set according to actual requirement(s).

The particle size of the erythritol crystal seeds refers to a parameter reflecting the particle size of the erythritol crystal seeds added into the second crystallization kettle 12. The particle size of the erythritol crystal seeds may be within a particle size range. In some embodiments, the particle size of the erythritol crystal seeds may be set according to actual requirement(s).

The crystal-cultivation time length of the second crystallization kettle 12 refers to a time length required for crystal cultivation in the second crystallization kettle 12. The crystal-cultivation time length of the second crystallization kettle 12 may be within a time range. In some embodiments, the crystal-cultivation time length of the second crystallization kettle may be set according to actual requirement(s). More descriptions regarding the crystal cultivation may be found in elsewhere in the present disclosure.

The evaporation crystallization time length of the second crystallization kettle 12 refers to a time length required for evaporation crystallization in the second crystallization kettle 12. The evaporative crystallization time length of the second crystallization kettle may be within a time range. In some embodiments, the evaporation crystallization time length of the second crystallization kettle may be set according to actual requirement(s).

The crystal cultivation has two advantages. Firstly, the relatively high supersaturation in the liquid system can be reduced with time, such that the crystallization process is performed at a controllable level of supersaturation. Secondly, the uniformity of the particle size can be improved. Since the crystallization process is a dynamic equilibrium of constant dissolution and crystallization, and a solubility of a crystal particle is related to a particle size of the crystal particle, a crystal particle with a small particle size has a higher and faster solubility than a crystal particle with a large particle size. With the prolongation of time, ripening occurs, small particles dissolve and disappear, and the particle size can be more uniform.

The crystal seeds are used to provide sites for crystal growth. In some embodiments, the addition of the erythritol crystal seeds may increase a crystallization rate, which facilitates the crystal growth.

The particle size refers to an indicator reflecting a size of particles, and the unit of the particle size is mesh. In a Taylor standard sieve, the mesh refers to a number of sieve holes in a 1-inch length. The larger the value of the mesh is, the finer the particle may be.

In some embodiments, in operation 2, when the crystallization is performed in the second crystallization kettle 12, a stirring speed is set to be within a range from 50 to 100 revolutions per minute (rpm), and a crystallization temperature is set to be within a range from 60 to 65° C. After the erythritol crystal seeds are added, and the erythritol crystal seeds are dispersed uniformly in a sugar liquid, the crystallization temperature and the stirring speed are maintained constant, and the crystallization is cultivated for 0.5 to 2 hours (h). After the cultivation of the crystallization, a pressure of the second crystallization kettle 12 is set to be within a range from 150 to 220 millibar (mbar), and an evaporation crystallization is performed on the sugar liquid while stirring. After the evaporation crystallization is performed for 2 to 6 hours, the evaporation crystallization is stopped and crystals are out of the second crystallization kettle 12. The addition ratio of the erythritol crystal seeds is within a range from 1 to 5%, and the particle size of the erythritol crystal seeds is within a range from 60 to 120 mesh.

The hot-air temperature in the hot-air drying tank 14 refers to a temperature parameter of the hot-air in the hot-air drying tank 14. The hot-air temperature in the hot-air drying tank may be within a temperature range. In some embodiments, the hot-air temperature in the hot-air drying tank 14 may be set according to actual requirement(s).

The cold-air temperature in the fluidized drying bed 15 refers to a temperature parameter of the cold-air in the fluidized drying bed 15. The cold-air temperature in the fluidized drying bed may be within a temperature range. In some embodiments, the cold-air temperature in the fluidized drying bed 15 may be set according to actual requirement(s).

In some embodiments, in operation 3, the hot-air temperature in the hot-air drying tank 14 is within a range from 90 to 100° C., and the cold-air temperature in the fluidized drying bed 15 is within a range from 15 to 30° C.

In some embodiments, the system for preparing the compound crystal of the erythritol and the high-intensity sweetener may further include a controller 110 and a processor 120.

The controller 110 refers to a device that controls the system to perform relevant operations based on predetermined programs or related instructions. In some embodiments, the controller 110 may include a programmable logic circuit (PLD), a microcontroller unit, etc. In some embodiments, the controller 110 may include an interactive device, such as, an interactive display. The predetermined program may be manually preset. The relevant instructions may be predetermined or manually input in real time through the interactive device.

The processor 120 may be configured to process data and/or information obtained from the controller 110 or the system. The processor 120 may execute programs or instructions based on such data, information, and/or processing results to perform one or more of the functions described in the present disclosure. In some embodiments, the processor 120 may include one or more sub-processing devices (e.g., a single-core processing device or a multi-core processing device). Merely by way of example, the processor 120 may include a central processing unit (CPU), an application-specific integrated circuit (ASIC), an application-specific instruction processor (ASIP), a graphics processor (GPU), a physical processor (PPU), a digital signal processor (DSP), a field programmable gate array (FPGA), a reduced instruction set computer (RISC), a microprocessor, etc., or any combination thereof.

In some embodiments, the controller 110 and the processor 120 may be integral. For example, the processor 120 may be a portion of the controller 110.

FIG. 2 is a flowchart illustrating an exemplary process for generating and sending preparation instructions according to some embodiments of the present disclosure. In some embodiments, process 200 may be executed by the controller 110 and the processor 120. As shown in FIG. 2, process 200 may include the following operations.

In 210, a taste requirement and a particle size distribution requirement of a compound crystallization product of erythritol and a high-intensity sweetener may be obtained through a controller (e.g., the controller 110).

The taste requirement refers to requirement intensities of different tastes of the compound crystallization product. The tastes may include sweetness, bitterness, metallic taste, delayed bitterness, delayed sweetness, acerbic taste, or the like, or any combination thereof. In some embodiments, the intensity (i.e., the requirement intensity) of each taste may be indicated by a numerical value. The larger the numerical value, the larger the intensity of the type of taste. For example, the intensity may be expressed as a numerical value within a range from 1 to 14. In some embodiments, the taste requirement may be expressed by a value of the intensity of each desired taste. For example, the taste requirement may be represented by a taste feature vector including a numerical value corresponding to each taste. For instance, a taste feature vector {6.5, 2.1, 1.8, 2.5, 1.7, 1.9} may indicate that an intensity of the sweetness is 6.5, an intensity of the bitter is 2.1, an intensity of the metallic taste is 1.8, an intensity of the delayed bitterness is 2.5, an intensity of the delayed sweetness is 1.7, and an intensity of the acerbic taste is 1.9.

According to some embodiments of the present disclosure, an intensity of a sweetness of a crystal in which a concentration of the sucrose is 7% is defaulted to 7. The intensities of the sweetness and other types of tastes may be expressed based on the default value. Since a target taste of the compound crystallization product is the sweetness, only the intensity of the sweetness is relatively high, and the other tastes are maintained at a relatively low level. The sweetness of the compound crystal may be 0.7 to 2 times that of the sucrose. In some embodiments, the controller 110 may set the intensity of each of the various types of taste to be within a range from 1 to 14.

The particle size distribution requirement refers to a requirement for a particle size distribution of the compound crystallization product. A particle size distribution refers to a distribution of a proportion of each particle size among different particle size ranges. In some embodiments, the particle size distribution requirement may be represented by percentages of crystals with particle size ranges in the compound crystallization product. For example, the particle size distribution requirement may be represented by a particle size distribution feature vector including the percentages of different particle sizes ranges. For instance, if the different particle sizes ranges include a range from 10 to 20 mesh, a range from 20 to 30 mesh, a range from 30 to 40 mesh, a range from 40 to 50 mesh, a range from 50 to 60 mesh, and a range less than 60 mesh, a particle size distribution feature vector {2, 3, 15, 40, 25, 15} may represent that a proportion of crystals whose particle size is within the range from 10 to 20 mesh in the compound crystallization product is 2%, a proportion of crystals whose particle size is within the range from 20 to 30 mesh in the compound crystallization product is 3%, a proportion of crystals whose particle size is within the range from 30 to 40 mesh in the compound crystallization product is 15%, a proportion of crystals whose particle size is within the range from 40 to 50 mesh in the compound crystallization product mesh is 40%, a proportion of crystals whose particle size is within the range from 50 to 60 mesh in the compound crystallization product is 25%, and a proportion of crystals whose particle size is within the range less than 60 mesh in the compound crystallization product is 15%.

In some embodiments, the controller 110 may obtain the taste requirement and the particle size distribution requirement through a variety of manners. For example, the controller 110 may obtain the taste requirement and the particle size distribution requirement through an interactive device. For instance, the taste requirement and the particle size distribution requirement may be manually inputted into the interactive device. As another example, the controller 110 may automatically determine a default taste requirement and a default particle size distribution requirement based on historical taste requirements and historical particle size distribution requirements.

In 220, setting values of preparation parameters may be determined based on the taste requirement and the particle size distribution requirement through a processor (e.g., the processor 120).

The setting values of the preparation parameters refer to values corresponding to the preparation parameters set in an actual preparation process. More descriptions regarding the preparation parameters and the setting values of the preparation parameters may be found in FIG. 1 and relevant descriptions thereof.

In some embodiments, the processor 120 may determine the setting values of the preparation parameters through a variety of manners based on the taste requirement and the particle size distribution requirement. For example, a vector database may be constructed based on historical data, a match may be performed in the vector database based on feature vectors, a reference vector with a highest similarity to the feature vector representing the taste requirement and the particle size distribution requirement may be determined, and reference setting values of the preparation parameters corresponding to the reference vector may be determined as current setting values of the preparation parameters.

The feature vectors may include the taste feature vector and the particle size distribution feature vector. The vector database may include reference vectors and reference setting values of the preparation parameters corresponding the reference vectors. The reference vectors may include historical taste feature vectors corresponding to the historical taste requirements and historical particle size distribution feature vectors corresponding to the historical particle size distribution requirements in the historical data, and the reference setting values of the preparation parameters corresponding the reference vectors may be determined based on historical setting values. The comprehensive similarity may be determined based on a first similarity between the taste feature vector and the historical taste feature vector, and a second similarity between the particle size distribution feature vector and the historical particle size distribution feature vector. For example, the comprehensive similarity may be a weighted average of the first similarity and the second similarity. The weights of the first similarity and the second similarity may be manually preset. In some embodiments, each of the first similarity and the second similarity may be determined based on a cosine distance, a Euclidean distance, etc.

In some embodiments, the processor 120 may also determine, based on the taste requirement and the particle size distribution requirement, the setting values of the preparation parameters through a parameter prediction model. More descriptions regarding the determination of the setting values of the preparation parameters through the parameter prediction model may be found in FIG. 3 and relevant descriptions thereof.

In 230, one or more preparation instructions may be generated based on the setting values of the preparation parameters, and the instructions may be sent to at least one of the first crystallization kettle 5, the blending tank 10, the second crystallization kettle 12, the hot-air drying tank 14, and the fluidized drying bed 15, respectively, through the controller (e.g., the controller 110).

A preparation instruction refers to an instruction issued to a corresponding device during the preparation process. In some embodiments, a device may correspond to a preparation instruction, and each preparation instruction may include only preparation parameter(s) related to the corresponding device. For example, if the preparation parameter(s) related to the first crystallization kettle 5 include a first crystallization temperature of the first crystallization kettle 5, the preparation instruction received by the first crystallization kettle 5 may include the first crystallization temperature of the first crystallization kettle 5.

More descriptions regarding the first crystallization temperature may be found in FIG. 1 and relevant descriptions thereof.

In some embodiments, the controller 110 may generate the one or more preparation instructions based on the setting values of the preparation parameters, and send the preparation instructions to the corresponding devices. For example, a preparation instruction corresponding to the first crystallization kettle 5 may be generated based on parameter(s) among the preparation parameter that relates to the first crystallization kettle 5, and the preparation instruction may be sent to the first crystallization kettle 5.

According to some embodiments of the present disclosure, by determining the setting values of the preparation parameters based on the taste requirement and the particle size distribution requirement, generating the one or more preparation instructions based on the setting values of the preparation parameters, and sending the one or more preparation instructions to the corresponding devices, more reasonable preparation parameters can be set based on the taste requirement and the particle size distribution requirement of the compound crystallization product, which improves the preparation efficiency and the product quality.

It should be noted that the descriptions of process 200 is merely provided for the purposes of illustration and explanation, and are not intended to limit the scope of the present disclosure. For persons having ordinary skills in the art, various variations and modifications to process 200 may be conducted under the guidance of the present disclosure. However, these variations and modifications may not depart from the scope of the present disclosure.

FIG. 3 is a schematic diagram illustrating an exemplary process for determining setting values of preparation parameters through a parameter prediction model according to some embodiments of the present disclosure.

In some embodiments, as shown in FIG. 3, the processor 120 may determine setting values 360 of preparation parameters based on a taste requirement 310 and a particle size distribution requirement 320 through a parameter prediction model 350. More descriptions regarding the taste requirement, the particle size distribution requirement, the setting values of the preparation parameters may be found in FIG. 2 and relevant descriptions thereof.

The parameter prediction model 350 refers to a model for predicting the setting values of the preparation parameters. In some embodiments, the parameter prediction model 350 may be a machine learning model. For example, the parameter prediction model 350 may include a neural network model (NN), a deep neural network model (DNN), or the like, or any combination thereof. In some embodiments, inputs of the parameter prediction model 350 may include a taste requirement and a particle size requirement (which may be in the form of feature vectors as described in FIG. 2), and outputs of the parameter prediction model 350 may be the setting values of the preparation parameters.

In some embodiments, the parameter prediction model 350 may be obtained based on parameter training samples with training labels. Each of the parameter training samples may include a sample taste and a sample particle size distribution of a sample compound crystallization product in a historical record, which may be obtained from historical data. The sample taste may correspond to a taste requirement inputted into the parameter prediction model 350, and the sample particle size distribution may correspond to a particle size distribution requirement inputted into the parameter prediction model 350. The training labels corresponding to the parameter training sample may be setting values of historical preparation parameters corresponding to the sample compound crystallization product. The training labels may be automatically labeled by the processor 120 based on the historical record.

In some embodiments, the sample taste may be a vector reflecting an actual taste of the sample compound crystallization product. The sample taste may be obtained based on a historical taste test result of the sample compound crystallization product. The sample particle size distribution may be a vector reflecting an actual particle size distribution of the sample compound crystallization product. The sample particle size distribution may be determined based on an actual particle size distribution of the sample compound crystallization product in the historical record. In some embodiments, the actual particle size distribution of the sample compound crystallization product in the historical record may be automatically obtained and stored by the preparation system through a relevant measuring instrument (e.g., a laser particle sizer, etc.).

In some embodiments, the processor 120 may input the parameter training samples into an initial parameter prediction model, determine a value of a loss function based on outputs of the initial parameter prediction model and the training labels indicating the setting values of the historical preparation parameters of the sample compound crystalline products, and iteratively update the initial parameter prediction model based on the value of the loss function. When a value of the loss function satisfies an iteration completion condition, the training may be completed and the trained parameter prediction model may be obtained. The iteration completion condition may include a loss function converges, the number of iterations reaches a threshold, etc.

According to some embodiments of the present disclosure, by training the initial parameter prediction model using the sample taste and sample particle size distribution of the sample compound crystallization product in the historical record as the parameter training sample, and using the setting values of the historical preparation parameters corresponding to the sample compound crystallization product as the training labels, the parameter prediction model may be obtained, which improves the accuracy of the determination of the setting values of the preparation parameters according to product requirements.

In some embodiments, the inputs to the parameter prediction model 350 may further include a type 330 of a high-intensity sweetener.

In some embodiments, the type of the high-intensity sweetener may include stevioside, mogroside, sucralose, acesulfame, aspartame, or the like, or any combination thereof. More descriptions regarding the type of the high-intensity sweetener may be found in FIG. 1 and relevant descriptions thereof.

In some embodiments, when the inputs of the parameter prediction model 350 include the type of the high-intensity sweetener, the type of the high-intensity sweetener of the sample compound crystallization product may be added for training the parameter prediction model, and the trained parameter prediction model may be obtained. The specific training process may be referred to the relevant description above.

Considering that the production processes and results corresponding to different high-intensity sweeteners are different, by adding the type of the high-intensity sweetener as a model input, the accuracy of the parameter prediction model in controlling the particle size distribution and the taste of the different types of compound crystals can be improved.

In some embodiments, the inputs to the parameter prediction model 350 may also include historical setting values 340 of the preparation parameters.

The historical setting values of the preparation parameters refer to setting values of the preparation parameters that have been used in the current preparation process. For example, in the preparation process of the compound crystals, if the current process proceeds to operation N of the preparation process, setting values of the preparation parameters used in operation 1, operation 2, . . . , and operation (N−1) are historical setting values of the preparation parameters.

In some embodiments, the processor 120 may use different preparation parameters at different stages, respectively. When the preparation parameters in the earlier stage are set incorrectly (i.e., the historical setting values of the preparation parameters are incorrect), or when a producer adjusts the taste requirement or the particle size distribution requirement during the preparation process, but the historical setting values of the preparation parameters correspond to the taste requirement and the particle size distribution requirement before the adjustment, the processor 120 may change the taste or the particle size distribution by adjusting the setting values of the preparation parameters.

In some embodiments, the processor 120 may determine whether the taste or the particle size distribution can be adjusted currently by adjusting setting values of later preparation parameters. For example, effects of setting values of the preparation parameters at different stages on the taste and the particle size distribution may be determined based on historical data, so as to determine whether the taste or the particle size distribution can be adjusted by adjusting the setting values of the later preparation parameters.

Merely by way of example, the setting values of the preparation parameters and the corresponding order may be represented by a parameter 1, a parameter 2, a parameter 3, . . . , a parameter n. For example, the parameter 1 may be the first crystallization temperature of the first crystallization kettle 5, which is the first preparation parameter to be set during the entire preparation process. More descriptions regarding the preparation process may be found in FIG. 1 and relevant descriptions thereof. The processor 120 may determine, based on the following operations, whether the taste or the particle size distribution can be adjusted by adjusting the setting values of the later preparation parameters.

In S1, the parameter 1 may be fixed, a plurality of parameter setting groups with different setting values of preparation parameters from the parameter 2 to the parameter n may be selected, taste test results and particle size distributions of compound crystals corresponding to the plurality of parameter setting groups may be obtained, and the following determinations may be performed.

1) If there are significant differences in both the taste and the particle size distribution among the plurality of parameter setting groups, the taste and the particle size distribution can be adjusted by adjusting the setting values of the later preparation parameters after the current parameter is fixed.

2) If there is only a significant difference in the taste and no significant difference in the particle size distribution among the plurality of parameter setting groups, the taste can be adjusted but the particle size distribution cannot be adjusted by adjusting the setting values of the later preparation parameters after the current parameter is fixed.

3) If there is only a significant difference in the particle size distribution and no significant difference in the taste among the plurality of parameter setting groups, the particle size distribution can be adjusted but the taste cannot be adjusted by adjusting the setting values of the later preparation parameters after the current parameter is fixed.

4) If there is no significant difference in the taste and the particle size distribution among the plurality of parameter setting groups, the taste and the particle size distribution cannot be adjusted by adjusting the setting values of the later preparation parameters after the current parameter is fixed.

In S2, the parameter 1 and the parameter 2 may be fixed, a plurality of parameter setting groups with different setting values of preparation parameters from the parameter 3 to the parameter n may be selected, taste test results and particle size distributions of compound crystals corresponding to the plurality of parameter setting groups may be obtained, and the determinations 1)˜4) may be repeated.

In S3, referring to operations S1 and S2, fixed parameters may be sequentially added and the determinations 1)˜4) may be repeated until the parameters 1 to n are all fixed, or there is no significant difference in the taste and the particle size distribution corresponding to each parameter setting group.

The significant difference refers to an intensity difference of at least one taste exceeds a first difference threshold, or a difference degree of the particle size distribution exceeds a second difference threshold. The difference degree may be determined based on a relative entropy (KL dispersion) between the particle size distributions. The first difference threshold and the second difference threshold may be manually preset. More descriptions regarding the intensity of the taste may be found in FIG. 2 and relevant descriptions thereof.

Through the above operations, a parameter X corresponding to the taste and a parameter Y corresponding to the particle size distribution may be determined. The parameter X refers to a last parameter that can have an effect on the taste. That is, if the parameter X has already been set, the taste can no longer be changed by adjusting the preparation parameters after the parameter X. Similarly, the parameter Y refers to a last parameter that can have an effect on the particle size distribution. That is, if the parameter Y has already been set, the particle size distribution can no longer be changed by adjusting the preparation parameters after the parameter Y.

In some embodiments, when the inputs of the parameter prediction model 350 need to introduce the historical setting values of the preparation parameters, the processor 120 may add the historical setting values of the sample preparation parameters of the sample compound crystallization products to the parameter training samples, and change the training labels to setting values of the sample preparation parameters adjusted at the later stage, such that the trained parameter prediction model is obtained. The specific training process may be referred to the relevant description above.

In some embodiments, the adjusted parameter training samples and the adjusted training labels may be obtained by the processor 120 in a following manner. When a compound crystallization product is prepared, setting values of the preparation parameters may be determined based on a predetermined taste requirement and a predetermined particle size distribution requirement. A plurality of compound crystallization products may be obtained by adjusting setting values of later preparation parameters (prior to the setting of the parameter X and/or the parameter Y) at different stages of each preparation process, and actual tastes and actual particle size distributions (different from the predetermined taste requirement and the predetermined particle size distribution requirement) of the compound crystallization products may be determined, respectively. Multiple sets of data may be obtained by changing the predetermined taste requirement and the predetermined particle size distribution requirement during the preparation process. The historical setting values of the preparation parameters before the adjustment, the actual taste, and the actual particle size distribution of a corresponding final product may be used as a parameter training sample, and the setting values of the later preparation parameters corresponding to the parameter training sample may be used as training labels corresponding to the parameter training sample.

According to some embodiments of the present disclosure, by introducing the historical setting values of the preparation parameters into the parameter prediction model, more reasonable setting values of the preparation parameters may be determined through the parameter prediction model for later adjustment in the event that the set values of the preparation parameters are set incorrectly in the early stage, so as to avoid waste of resources, reduce losses, and improve production efficiency.

The system and the method for preparing the compound crystal of the erythritol and the high-intensity sweetener in the present disclosure are further described below according to specific embodiments.

### Embodiment 1

The embodiment 1 of the process for preparing the compound crystal of the erythritol and the high-intensity sweetener of the present disclosure may be performed the processor 120. The method may include the following operations.

(11) A sterilized erythritol fermentation liquid sequentially passed through the ceramic membrane filtration system 2 and the nanofiltration system 3, and a nanofiltrate with an erythritol content of 93% and a light transmittance of 85% was obtained by removing the bacteria and other impurities. The nanofiltrate was concentrated through the first evaporator 4 to obtain an erythritol concentrate with a refractive index of 55%. The erythritol concentrate was evaporated and crystallized in the first crystallization kettle 5 to obtain an erythritol sugar paste. A crystallization temperature of the first crystallization kettle 5 was 60° C. The erythritol sugar paste was centrifuged through the first centrifuge 6 to obtain an erythritol monocrystalline sugar and a primary centrifugation mother liquor. The primary centrifugation mother liquor was returned for nanofiltration and then reused. That is, the primary centrifugation mother liquor was imported into the nanofiltration system 3, the primary centrifugation mother liquor and the filtrate in the nanofiltration system 3 were mixed to obtain a mixed solution, and then the mixed solution was returned to the first evaporator 4 through the pipeline for subsequent operations.

(12) The erythritol monocrystalline sugar was introduced into the dissolving sugar tank 7 for dissolution, and a dissolved liquid was obtained. The dissolved liquid sequentially passed through the decolorization tank 8 and the ion exchange column 9 to obtain a decolorization separation liquid with a light transmittance of 98% and a conductivity of 18 μs/cm. A blending liquid was obtained by mixing the decolorization separation liquid and a stevioside liquid in the blending tank 10 at a blending proportion. An erythritol content in the blending liquid is 99.84%, and a stevioside content in the blending liquid is 0.1%. The blending liquid was introduced into the second evaporator 11 for the concentration to obtain a blending concentrate of the erythritol and the stevioside with a refractive index of 66%.

(13) The blending concentrate was introduced into the second crystallization kettle 12. A stirring speed was set to 60 rpm, a temperature of the second crystallization kettle 12 was set to 65° C. Erythritol crystal seeds were added into the second crystallization kettle 12. An addition ratio of the erythritol crystal seeds was 2%, and a particle size of the erythritol crystal seeds was within a range from 60 to 80 mesh. After the erythritol crystal seeds were dispersed uniformly in a sugar liquid, the temperature and stirring speed were maintained constant, and the crystallization was cultivated for 1 h. After the cultivation of the crystallization, a pressure of the second crystallization kettle 12 was set to 18 Mpa, and an evaporation crystallization was performed on the sugar liquid while stirring. When the evaporation crystallization was performed for 4 h, the crystallization was stopped, and crystals were out of the second crystallization kettle 12 to obtain a compound crystallization sugar paste of the erythritol and the stevioside.

(14) The compound crystallization sugar paste of the erythritol and the stevioside was separated by the second centrifuge 13 to obtain compound crystals of the erythritol and the stevioside and a secondary mother liquor. Compound crystallization products of the erythritol and the stevioside were obtained by performing a hot-air drying operation at 90° C. and a cold-air drying operation at 30° C. on the compound crystals, and the secondary mother liquor (or the secondary centrifugal mother liquor) was returned to the blending tank 10 for reuse.

A particle size distribution of the compound crystallization products was obtained by sieving the compound crystallization products of the erythritol and the stevioside obtained in embodiment 1. The particle size distribution may be shown in Table 1.

TABLE 1
Particle Size Distribution of Compound Crystallization
Products of Erythritol and Stevioside of Embodiment 1

| | Particle size | | | | | |
|---|---|---|---|---|---|---|
| | | | | | | Less |
| | | | | | | than |
| | 10~20 | 20~30 | 30~40 | 40~50 | 50~60 | 60 |
| | mesh | mesh | mesh | mesh | mesh | mesh |
| Percentage/% | 42.68 | 28.01 | 17.43 | 8.38 | 1.49 | 2.01 |

As Table 1, the percentage of the compound crystallization products of the erythritol and the stevioside with the particle size from 10 to 30 mesh was 70.69%.

### Embodiment 2

The embodiment 2 of the process for preparing the compound crystal of the erythritol and the high-intensity sweetener of the present disclosure may include the following operations.

(21) A sterilized erythritol fermentation liquid sequentially passed through the ceramic membrane filtration system 2 and the nanofiltration system 3, and a nanofiltrate with an erythritol content of 96% and a light transmittance of 87% was obtained by removing the bacteria and other impurities. The nanofiltrate was concentrated through the first evaporator 4 to obtain an erythritol concentrate with a refractive index of 60%.

The erythritol concentrate was evaporated and crystallized in the first crystallization kettle 5 to obtain an erythritol sugar paste. A crystallization temperature of the first crystallization kettle 5 was 60° C. The erythritol sugar paste was centrifuged through the first centrifuge 6 to obtain an erythritol monocrystalline sugar and a primary centrifugation mother liquor. The primary centrifugation mother liquor was returned for nanofiltration and then reused. That is, the primary centrifugation mother liquor was imported into the nanofiltration system 3, the primary centrifugation mother liquor and the filtrate in the nanofiltration system 3 were mixed to obtain a mixed solution, and then the mixed solution was returned to the first evaporator 4 through the pipeline for subsequent operations.

(22) The erythritol monocrystalline sugar was introduced into the dissolving sugar tank 7 for dissolution, and a dissolved liquid was obtained. The dissolved liquid sequentially passed through the decolorization tank 8 and the ion exchange column 9 to obtain a decolorization separation liquid with a light transmittance of 99% and a conductivity of 25 μs/cm. A blending liquid was obtained by mixing the decolorization separation liquid and a stevioside liquid in the blending tank 10 at a blending proportion. An erythritol content in the blending liquid is 99.11%, a stevioside content in the blending liquid is 0.64%, and a mogroside content in the blending liquid is 0.25%. The blending liquid was introduced into the second evaporator 11 for the concentration to obtain a blending concentrate of the erythritol and the stevioside with a refractive index of 70%.

(23) The blending concentrate was introduced into the second crystallization kettle 12. A stirring speed was set to 100 rpm, a temperature of the second crystallization kettle 12 was set to 64° C. Erythritol crystal seeds were added into the second crystallization kettle 12. An addition ratio of the erythritol crystal seeds was 5%, and a particle size of the erythritol crystal seeds was within a range from 100 to 120 mesh. After the erythritol crystal seeds were dispersed uniformly in a sugar liquid, the temperature and stirring speed were maintained constant, and the crystallization was cultivated for 0.5 h. After the cultivation of the crystallization, a pressure of the second crystallization kettle 12 was set to 20 Mpa, and an evaporation crystallization was performed on the sugar liquid while stirring. When the evaporation crystallization was performed for 6 h, the crystallization was stopped, and crystals were out of the second crystallization kettle 12 to obtain a compound crystallization sugar paste of the erythritol and the stevioside.

(24) The compound crystallization sugar paste of the erythritol and the stevioside was separated by the second centrifuge 13 to obtain compound crystals of the erythritol and the stevioside and a secondary mother liquor. Compound crystallization products of the erythritol and the stevioside were obtained by performing a hot-air drying operation at 95° C. and a cold-air drying operation at 22° C. on the compound crystals, and the secondary mother liquor (or the secondary centrifugal mother liquor) was returned to the blending tank 10 for reuse.

A particle size distribution of the compound crystallization products was obtained by sieving the compound crystallization products of the erythritol and the stevioside obtained in embodiment 2. The particle size distribution may be shown in Table 2.

TABLE 2
Particle Size Distribution of Compound Crystallization
Products of Erythritol and Stevioside of Embodiment 2

| | Particle size | | | | | |
|---|---|---|---|---|---|---|
| | | | | | | Less |
| | | | | | | than |
| | 10~20 | 20~30 | 30~40 | 40~50 | 50~60 | 60 |
| | mesh | mesh | mesh | mesh | mesh | mesh |
| Percentage/% | 1.5 | 3.68 | 18.62 | 38.83 | 23.24 | 14.12 |

As Table 2, the percentage of the compound crystallization products of the erythritol and the stevioside with the particle size less than 40 mesh was 76.2%.

### Embodiment 3

The embodiment 3 of the process for preparing the compound crystal of the erythritol and the high-intensity sweetener of the present disclosure may include the following operations.

(31) A sterilized erythritol fermentation liquid sequentially passed through the ceramic membrane filtration system 2 and the nanofiltration system 3, and a nanofiltrate with an erythritol content of 95% and a light transmittance of 90% was obtained by removing the bacteria and other impurities. The nanofiltrate was concentrated through the first evaporator 4 to obtain an erythritol concentrate with a refractive index of 58%. The erythritol concentrate was evaporated and crystallized in the first crystallization kettle 5 to obtain an erythritol sugar paste. A crystallization temperature of the first crystallization kettle 5 was 65° C. The erythritol sugar paste was centrifuged through the first centrifuge 6 to obtain an erythritol monocrystalline sugar and a primary centrifugation mother liquor. The primary centrifugation mother liquor was returned for nanofiltration and then reused. That is, the primary centrifugation mother liquor was imported into the nanofiltration system 3, the primary centrifugation mother liquor and the filtrate in the nanofiltration system 3 were mixed to obtain a mixed solution, and then the mixed solution was returned to the first evaporator 4 through the pipeline for subsequent operations.

(32) The erythritol monocrystalline sugar was introduced into the dissolving sugar tank 7 for dissolution, and a dissolved liquid was obtained. The dissolved liquid sequentially passed through the decolorization tank 8 and the ion exchange column 9 to obtain a decolorization separation liquid with a light transmittance of 99% and a conductivity of 20 μs/cm. A blending liquid was obtained by mixing the decolorization separation liquid and a stevioside liquid in the blending tank 10 at a blending proportion. An erythritol content in the blending liquid is 95.00%, a sucralose content in the blending liquid is 0.88%, a stevioside content in the blending liquid is 1.12%, and a mogroside content in the blending liquid is 3.0%. The blending liquid was introduced into the second evaporator 11 for the concentration to obtain a blending concentrate of the erythritol and the stevioside with a refractive index of 68%.

(33) The blending concentrate was introduced into the second crystallization kettle 12. A stirring speed was set to 85 rpm, a temperature of the second crystallization kettle 12 was set to 65° C. Erythritol crystal seeds were added into the second crystallization kettle 12. An addition ratio of the erythritol crystal seeds was 1%, and a particle size of the erythritol crystal seeds was within a range from 80 to 100 mesh. After the erythritol crystal seeds were dispersed uniformly in a sugar liquid, the temperature and stirring speed were maintained constant, and the crystallization was cultivated for 2 h. After the cultivation of the crystallization, a pressure of the second crystallization kettle 12 was set to 15 Mpa, and an evaporation crystallization was performed on the sugar liquid while stirring. When the evaporation crystallization was performed for 2 h, the crystallization was stopped, and crystals were out of the second crystallization kettle 12 to obtain a compound crystallization sugar paste of the erythritol and the stevioside.

(34) The compound crystallization sugar paste of the erythritol and the stevioside was separated by the second centrifuge 13 to obtain compound crystals of the erythritol and the stevioside and a secondary mother liquor. Compound crystallization products of the erythritol and the stevioside were obtained by performing a hot-air drying operation at 100° C. and a cold-air drying operation at 15° C. on the compound crystals, and the secondary mother liquor (or the secondary centrifugal mother liquor) was returned to the blending tank 10 for reuse.

A particle size distribution of the compound crystallization products was obtained by sieving the compound crystallization products of the erythritol and the stevioside obtained in embodiment 3. The particle size distribution may be shown in Table 3.

TABLE 3
Particle Size Distribution of Compound Crystallization
Products of Erythritol and Stevioside of Embodiment 3

| | Particle size | | | | | |
|---|---|---|---|---|---|---|
| | | | | | | Less |
| | | | | | | than |
| | Larger | 20~30 | 30~40 | 40~50 | 50~60 | 60 |
| | than 20 | mesh | mesh | mesh | mesh | mesh |
| Percentage/% | 2.42 | 5.76 | 27.92 | 40.01 | 8.49 | 15.40 |

As Table 3, the percentage of the compound crystallization products of the erythritol and the stevioside with the particle size from 30 to 50 mesh was 67.93%.

More descriptions regarding the preparation parameters involved in the operations of the above embodiments and related terms may be found in FIG. 1 and relevant descriptions thereof.

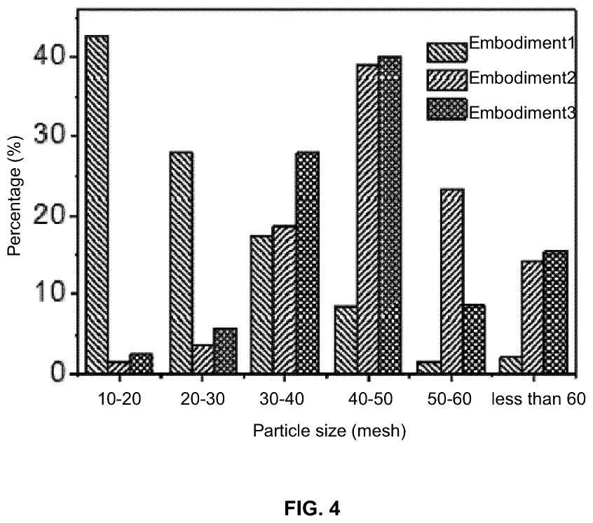

A particle size distribution diagram shown in FIG. 4 can be obtained by collating the particle size distributions of the compound crystallization products of the erythritol and the stevioside prepared in embodiments 1 to 3. FIG. 4 is a schematic diagram illustrating a particle size distribution for preparing a compound crystallization product of erythritol and a high-intensity sweetener according to some embodiments of the present disclosure.

A taste test was performed on the compound crystallized products of the erythritol and the stevioside obtained according to embodiments 1-3, respectively. Taste comparison tests were also performed on the compound crystallized products of the erythritol and the stevioside obtained according to embodiments 1-3 with sucrose products and stevioside products. Each of the sucrose products and the stevioside products was configured to a sugar liquid with a concentration of 7%. An intensity of a sweetness of a crystal in which a concentration of the sucrose is 7% was defaulted to 7. In some embodiments, the intensity of each taste can be indicated by a numerical value. The larger the numerical value, the larger the intensity of the type of taste. For example, the intensity of the taste can include 7 grades, wherein a value “1” represents very weak, a value “2” represents relatively weak, a value “3” represents-somewhat weak, a value “4” represents average, a value “5” represents somewhat strong, a value “6” represents relatively strong, and a value “7” represents very strong. Test results were obtained as shown in Table 4.

TABLE 4
Taste Test Results For Products

| | | | Embodi- | Embodi- | Embodi- |
|---|---|---|---|---|---|
| Items | Sucrose | Stevioside | ment 1 | ment 2 | ment 3 |
| Sweetness | 7 | 4.9 | 6.2 | 7.3 | 8.9 |
| Bitterness | 1.7 | 2.3 | 2.1 | 1.8 | 1.9 |
| Metallic | 1 | 1.4 | 1.5 | 1.2 | 1.4 |
| Taste | | | | | |
| Delayed | 2.5 | 1.8 | 2.7 | 2.8 | 3.1 |
| Sweetness | | | | | |
| Delayed | 1.7 | 1.9 | 1.6 | 1.6 | 1.8 |
| Bitterness | | | | | |
| Acerbic | 1.9 | 2.1 | 2.0 | 1.9 | 2.3 |
| Taste | | | | | |

The present disclosure also provides a non-transitory computer-readable storage medium storing computer instructions. When a computer reads the computer instructions from the storage medium, the computer may execute the method for preparing the compound crystal of the erythritol and the high-intensity sweetener.

The basic concepts have been described above, and it is apparent that to a person skilled in the art, the above detailed disclosure is intended as an example only and does not constitute a limitation of the present disclosure. Although not expressly stated herein, various modifications, improvements, and amendments may be made to the present disclosure by those skilled in the art. Such modifications, improvements, and amendments are suggested in the present disclosure, so such modifications, improvements, and amendments remain within the spirit and scope of the exemplary embodiments of the present disclosure.

Also, the present disclosure uses specific words to describe the embodiments of the present disclosure. For example, “an embodiment,” “one embodiment,” and/or “some embodiments” are meant to refer to a certain feature, structure, or characteristic associated with at least one embodiment of the present disclosure. Accordingly, it should be emphasized and noted that “an embodiment,” “one embodiment,” or “an alternative embodiment” mentioned two or more times in different places in the present disclosure do not necessarily refer to the same embodiment. Furthermore, certain features, structures, or characteristics in one or more embodiments of the present disclosure may be suitably combined.

Furthermore, unless explicitly stated in the claims, the use of order, numbers, letters, or other names for processing elements and sequences is not intended to limit the order of the processes and methods of the present disclosure. While various examples have been discussed in the disclosure as currently considered useful embodiments of the present disclosure, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or a mobile device.

Similarly, it should be noted that, for the sake of simplifying the presentation of the embodiments disclosed in the present disclosure and aiding in understanding one or more embodiments of the present disclosure, various features are sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately,” or “generally”. Unless otherwise stated, “about,” “approximately,” or “generally” indicates that a variation of +20% is permitted in the described numbers. Accordingly, in some embodiments, the numerical parameters used in the disclosure and claims are approximations, which may change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A system for preparing a compound crystal of erythritol and a high-intensity sweetener, wherein the system comprises a fermentation liquid storage tank, a ceramic membrane filtration system, a nanofiltration system, a first evaporator, a first crystallization kettle, a first centrifuge, a sugar dissolution tank, a decolorization tank, an ion exchange column, a blending tank, a second evaporator, a second crystallization kettle, a second centrifuge, a hot-air drying tank, a fluidized drying bed, and a finished product tank sequentially connected through pipelines, and
the system further comprises a high-intensity sweetener storage tank, a primary mother liquor tank, and a secondary mother liquor tank, wherein
the fermentation liquid storage tank is disposed with a feed inlet for sterilized erythritol fermentation liquid,
the nanofiltration system is disposed with a penetrating liquid feed outlet,
the penetrating liquid feed outlet is connected to a feed inlet of the first evaporator through a pipeline,
the sugar dissolution tank is disposed with a feed inlet for purified water,
the blending tank is disposed with a feed inlet for the high-intensity sweetener,
each of the first centrifuge and the second centrifuge is disposed with a solid material feed outlet and a liquid material feed outlet,
the liquid material feed outlet of the first centrifuge is connected to a feed inlet of the primary mother liquor tank through a pipeline,
a feed outlet of the primary mother liquor tank is connected to the feed inlet of the first evaporator through a pipeline,
the liquid material feed outlet of the second centrifuge is connected to a feed inlet of the secondary mother liquor tank through a pipeline,
a feed outlet of the secondary mother liquor tank is connected to a feed inlet of the blending tank through a pipeline, and
the finished product tank stores a prepared compound crystallization product of the erythritol and the high-intensity sweetener.

2. The system of claim 1, wherein the high-intensity sweetener includes at least one of stevioside, mogroside, sucralose, acesulfame, or aspartame.

3. The system of claim 1, further comprising a controller and a processor; wherein
the controller is configured to obtain a taste requirement and a particle size distribution requirement of the compound crystallization product of the erythritol and the high-intensity sweetener;
the processor is configured to determine, based on the taste requirement and the particle size distribution requirement, setting values of preparation parameters; and
the controller is further configured to generate, based on the setting values of the preparation parameters, one or more preparation instructions and send the one or more preparation instructions to at least one of the first crystallization kettle, the blending tank, the second crystallization kettle, the hot-air drying tank, and the fluidized drying bed, respectively, the preparation parameters including at least one of a first crystallization temperature of the first crystallization kettle, a blending ratio of the blending tank, a stirring speed of the second crystallization kettle, a second crystallization temperature of the second crystallization kettle, a pressure of the second crystallization kettle, an addition ratio and a crystal seed particle size of erythritol crystal seeds in the second crystallization kettle, a crystal-cultivation time length and an evaporation-crystallization time length in the second crystallization kettle, a hot-air temperature in the hot-air drying tank, or a cold-air temperature in the fluidized drying bed.

4. The system of claim 3, wherein the processor is further configured to:
determine, based on the taste requirement and the particle size distribution requirement, the setting values of the preparation parameters through a parameter prediction model, the parameter prediction model being a machine learning model.

5. The system of claim 4, wherein an input of the parameter prediction model include a type of the high-flavor sweetener.

6. The system of claim 4, wherein an input of the parameter prediction model include historical setting values of the preparation parameters.

7. A method for preparing a compound crystal of erythritol and a high-intensity sweetener, wherein the method uses the system according to claim 1, the method comprising:
operation 1, obtaining a penetrating liquid by sequentially conveying the sterilized erythritol fermentation liquid stored in the fermentation liquid storage tank to the ceramic membrane filtration system for filtration and the nanofiltration system for separation, and obtaining a solid erythritol monocrystalline sugar and a liquid primary mother liquor, respectively, by conveying the penetrating liquid to the first evaporator for concentration, the first crystallization kettle for crystallization, and the first centrifuge for centrifugation through the pipelines, wherein
the primary mother liquor is temporarily stored in the primary mother liquor tank and then returned to the first evaporator through the pipeline for reuse, and
in operation 1, a content of the erythritol in the penetrating liquid after the separation performed by the nanofiltration system is larger than 93%, and a light transmittance of the erythritol in the penetrating liquid is larger than 85%;
operation 2, obtaining a dissolved liquid by dissolving the erythritol monocrystalline sugar in the dissolving sugar tank, performing a decolorization operation through the decolorization tank and an impurity removal operation through the ion exchange column on the dissolved liquid sequentially, obtaining a blending liquid by mixing the processed dissolved liquid with the high-intensity sweetener liquid conveyed by the high-intensity sweetener storage tank and a secondary mother liquor conveyed by the secondary mother liquor tank, and then obtaining a solid compound moist sugar of the erythritol and the high-intensity sweetener, and the liquid secondary mother liquor, respectively, by sequentially introducing the blending liquid into the second evaporator for concentration, the second crystallization kettle for crystallization, and the second centrifuge for centrifugal separation through the pipelines, wherein
the secondary mother liquor is temporarily stored in the secondary mother liquor tank and then returned to the blending tank through the pipeline for reuse,
in operation 2, a light transmittance of the processed dissolved liquid is larger than 97% and a conductivity of the processed dissolved liquid is less than 30 microsecond per centimeter (μs/cm),
a refractive index of a blending concentrate after the concentration is performed on the blending liquid by the second evaporator is within a range from 65 to 70%, an erythritol content of the blending concentrate is within a range from 95 to 99.9%, and a high-intensity sweetener content of the blending concentrate is within a range from 0.1 to 5%; and
operation 3, obtaining the prepared compound crystallization product of the erythritol and the high-intensity sweetener temporarily stored in the finished product tank by sequentially performing a hot-air drying operation through the hot-air drying tank and a cold-air drying operation through the fluidized drying bed on the obtained compound moist sugar of the erythritol and the high-intensity sweetener through the pipelines.

8. The method of claim 7, further comprising:
obtaining a taste requirement and a particle size distribution requirement of the compound crystallization product of the erythritol and the high-intensity sweetener through a controller;
determining, based on the taste requirement and the particle size distribution requirement, setting values of preparation parameters through a processor, the preparation parameters including at least one of a first crystallization temperature of the first crystallization kettle, a blending ratio of the blending tank, a stirring speed of the second crystallization kettle, a second crystallization temperature of the second crystallization kettle, a pressure of the second crystallization kettle, an addition ratio and a crystal seed particle size of erythritol crystal seeds in the second crystallization kettle, a crystal-cultivation time length and an evaporation-crystallization time length in the second crystallization kettle, a hot-air temperature in the hot-air drying tank, or a cold-air temperature in the fluidized drying bed.

9. The method of claim 8, wherein the obtaining a taste requirement and a particle size distribution requirement of the compound crystallization product of the erythritol and the high-intensity sweetener through a controller includes:
determining, based on the taste requirement and the particle size distribution requirement, the setting values of the preparation parameters through a parameter prediction model, the parameter prediction model being a machine learning model.

10. The method of claim 9, wherein an input of the parameter prediction model include a type of the high-flavor sweetener.

11. The method of claim 9, wherein an input of the parameter prediction model include historical setting values of the preparation parameters.

12. The method of claim 7, wherein in operation 1, a refractive index of an erythritol concentrate obtained after the concentration is performed on the penetrating liquid by the first evaporator is within a range from 55 to 60%, and a crystallization temperature of the first crystallization kettle is within a range from 60 to 65° C.

13. The method of claim 7, wherein in operation 2,
when the crystallization is performed in the second crystallization kettle, a stirring speed is set to be within a range from 50 to 100 revolutions per minute (rpm), and a crystallization temperature is set to be within a range from 60 to 65° C.;
after erythritol crystal seeds are added, and the erythritol crystal seeds are dispersed uniformly in a sugar liquid, the crystallization temperature and the stirring speed are maintained constant, and the crystallization is cultivated for 0.5 to 2 hours (h);
after the cultivation of the crystallization, a pressure of the second crystallization kettle is set to be within a range from 150 to 220 millibar (mbar), and an evaporation crystallization is performed on the sugar liquid while stirring,
after the evaporation crystallization is performed for 2 to 6 hours, the evaporation crystallization is stopped and crystals are out of the second crystallization kettle, an addition ratio of the erythritol crystal seeds being within a range from 1 to 5%, and a particle size of the erythritol crystal seeds being within a range from 60 to 120 mesh.

14. The method of claim 7, wherein in operation 3, a hot-air temperature in the hot-air drying tank is within a range from 90 to 100° C., and a cold-air temperature in the fluidized drying bed is within a range from 15 to 30° C.

15. A non-transitory computer-readable storage medium storing computer instructions, wherein when a computer reads the computer instructions from the storage medium, the computer executes a method for preparing a compound crystal of erythritol and a high-intensity sweetener, wherein the method uses the system according to claim 1, the method comprising:
operation 1, obtaining a penetrating liquid by sequentially conveying the sterilized erythritol fermentation liquid stored in the fermentation liquid storage tank to the ceramic membrane filtration system for filtration and the nanofiltration system for separation, and obtaining a solid erythritol monocrystalline sugar and a liquid primary mother liquor, respectively, by conveying the penetrating liquid to the first evaporator for concentration, the first crystallization kettle for crystallization, and the first centrifuge for centrifugation through the pipelines, wherein
the primary mother liquor is temporarily stored in the primary mother liquor tank and then returned to the first evaporator through the pipeline for reuse, and
in operation 1, a content of the erythritol in the penetrating liquid after the separation performed by the nanofiltration system is larger than 93%, and a light transmittance of the erythritol in the penetrating liquid is larger than 85%;
operation 2, obtaining a dissolved liquid by dissolving the erythritol monocrystalline sugar in the dissolving sugar tank, performing a decolorization operation through the decolorization tank and an impurity removal operation through the ion exchange column on the dissolved liquid sequentially, obtaining a blending liquid by mixing the processed dissolved liquid with the high-intensity sweetener liquid conveyed by the high-intensity sweetener storage tank and a secondary mother liquor conveyed by the secondary mother liquor tank, and then obtaining a solid compound moist sugar of the erythritol and the high-intensity sweetener, and the liquid secondary mother liquor, respectively, by sequentially introducing the blending liquid into the second evaporator for concentration, the second crystallization kettle for crystallization, and the second centrifuge for centrifugal separation through the pipelines, wherein
the secondary mother liquor is temporarily stored in the secondary mother liquor tank and then returned to the blending tank through the pipeline for reuse,
in operation 2, a light transmittance of the processed dissolved liquid is larger than 97% and a conductivity of the processed dissolved liquid is less than 30 microsecond per centimeter (μs/cm),
a refractive index of a blending concentrate after the concentration is performed on the blending liquid by the second evaporator is within a range from 65 to 70%, an erythritol content of the blending concentrate is within a range from 95 to 99.9%, and a high-intensity sweetener content of the blending concentrate is within a range from 0.1 to 5%; and
operation 3, obtaining the prepared compound crystallization product of the erythritol and the high-intensity sweetener temporarily stored in the finished product tank by sequentially performing a hot-air drying operation through the hot-air drying tank and a cold-air drying operation through the fluidized drying bed on the obtained compound moist sugar of the erythritol and the high-intensity sweetener through the pipelines.
